(19) United States
(12) Patent Application Publication
(10) Pub. No.: US 2001/0051613 A1
(43) Pub. Date: Dec. 13, 2001

(54) Novel formulations of fexofenadine

(75) Inventors: Lisbeth Illum, Nottingham (GB); Peter James Watts, Nottingham (GB); Yu-Hui Cheng, Nottingham (GB)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: West Pharmaceutical Services Drug Delivery & Clinical Research Centre, Ltd.

(21) Appl. No.: 09/834,312

(22) Filed: Apr. 13, 2001

Related U.S. Application Data
(63) Continuation of application No. PCT/GB99/03396, filed on Oct. 12, 1999.

(30) Foreign Application Priority Data
Oct. 13, 1998 (GB) 9822170.8

(51) Int. Cl. A61K 31/718

(52) U.S. Cl. 514/58

(57) ABSTRACT

The present invention provides a composition comprising (i) fexofenadine or a pharmaceutically acceptable salt thereof and (ii) a pharmaceutical excipient that increases the solubility of the fexofenadine or salt in water. The pharmaceutical excipient is preferably a cyclodextrin.

### CROSS-REFERENCE TO RELATED APPLICATION

This application is a continuation of International Application No. PCT/GB99/03396, filed Oct. 12, 1999, the disclosure of which is incorporated herein by reference.

### BACKGROUND OF THE INVENTION

The present invention relates generally to a formulation of fexofenadine and particularly to a liquid formulation of fexofenadine. More specifically, the present invention relates to aqueous formulations of fexofenadine which are suitable for nasal or ophthalmic administration.

Fexofenadine is a H₁-histamine antagonist drug, which has been recently introduced for relief of the symptoms of allergy. The drug is the active metabolite of another antihistamine, terfenadine. High plasma concentrations of terfenadine have been associated with rare incidences of cardiac arrhythmias and the drug is gradually being withdrawn from clinical use, with fexofenadine being promoted as a replacement.

To date only oral formulations of fexofenadine have been developed. However, nasal formulations of the drug for local treatment of allergic rhinitis would be advantageous. A particularly desirable nasal formulation for local action would be one having prolonged retention in the nasal cavity by the use of a gelling and/or bioadhesive liquid or powder formulation. A liquid formulation of fexofenadine adapted for nasal administration may also be appropriate for ophthalmic administration, although the range of excipients suitable for administration into the eye is more limited, in part because the eye has greater sensitivity than the nasal cavity.

Fexofenadine is used in the form of the pharmaceutically acceptable hydrochloride salt (MW 538).

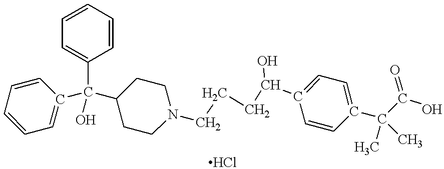

### Fexofenadine Hydrochloride

Fexofenadine hydrochloride shows highest water solubility between pH 2 and 3 and above pH 9. For use in the nasal cavity and the eye a pH in the range 4 to 8 should be chosen to prevent possible irritation. However, the solubility of the anhydrous form of fexofenadine hydrochloride between pH 4 and 9 is low, for example around 0.2 to 0.5 mg/ml.

A nasal dose for fexofenadine has not been established. However, based on a daily oral dose of 120 mg and the nasal/oral dose ratio for other antihistamines, a nasal fexofenadine dose in the range 1 to 5 mg/nostril can be assumed. Therefore, for a liquid formulation, with a 0.1 ml dose volume, a concentration of 10 to 50 mg/ml fexofenadine would be required.

The major challenge to the development of a nasal or ophthalmic formulation of fexofenadine hydrochloride is the limited solubility of the drug.

### BRIEF SUMMARY OF THE INVENTION

The present applicant has developed a formulation comprising fexofenadine or a pharmaceutically acceptable salt thereof which is within the pH range suitable for nasal or ophthalmic administration. This formulation comprises a pharmaceutical excipient, such as a cyclodextrin, which is able to increase the solubility of fexofenadine or its pharmaceutically acceptable salts in water. The formulation may also provide for the controlled release of the fexofenadine or a pharmaceutically acceptable salt thereof in the nasal cavity.

According to the present invention, there is provided a composition comprising (i) fexofenadine or a pharmaceutically acceptable salt thereof and (ii) a pharmaceutical excipient which increases the solubility of the fexofenadine or salt in water.

The composition is preferably adapted for nasal or ophthalmic administration and, accordingly, in a preferred embodiment, the present invention provides a nasally or ophthalmically administrable composition.

The composition of the invention may be a solid, e.g. a microsphere system, but is preferably a liquid composition and more preferably is aqueous. The aqueous composition may be a solution, suspension or an emulsion.

Accordingly, in a preferred aspect of the present invention, there is provided an aqueous composition comprising (i) fexofenadine or a pharmaceutically acceptable salt thereof, (ii) a pharmaceutical excipient which increases the solubility of the fexofenadine or salt in water, and (iii) an aqueous vehicle, e.g. water.

The water should, of course, be of pharmaceutically acceptable purity.

Suitable pharmaceutically acceptable salts of fexofenadine include the hydrochloride, hydrobromide, acetate, mesylate and sulphate salts. An especially preferred salt is the hydrochloride salt. The base of fexofenadine can also be used.

Hereinafter, the term fexofenadine refers collectively to both fexofenadine and its pharmaceutically acceptable salts unless the context requires otherwise.

The concentration of fexofenadine in a liquid composition can be from 100 μg/ml to 100 mg/ml. A preferred concentration range is 1 to 75 mg/ml and an especially preferred concentration range is 10 to 50 mg/ml.

The concentration of fexofenadine in a solid formulation can be from 0.5 to 40% w/w. A preferred concentration range is 1 to 30% w/w and an especially preferred concentration range is 2 to 20% w/w.

Suitable pharmaceutical excipients which increase the solubility of the fexofenadine or salt in water include pharmaceutically acceptable, water miscible solvents such as propylene glycol and glycofurol (tetraglycol). Other suitable excipients include those materials which are able to complex with the fexofenadine.

Especially preferred pharmaceutical excipients for enhancing the solubility of the fexofenadine or salt in water are the cyclodextrins.

Cyclodextrins (CD) are industrially produced cyclic oligosaccharides which comprise glucopyranose units. The three major cyclodextrins are α, β and γ cyclodextrin which comprise 6, 7 and 8 glucopyranose units respectively. The physicochemical properties of α, β and γ cyclodextrins are different and they have different solubilities in water.

As well as the α, β and γ cyclodextrins, suitable cyclodextrin excipients for use in the present invention include the derivatised cyclodextrins, such as the alkyl and alkoxy substituted cyclodextrins. Preferred derivatives are the derivatives of β-cyclodextrins, such as the dimethyl-β-cyclodextrins, e.g. 2,6-dimethyl 14-β-cyclodextrin, trimethyl-β-cylodextrins, e.g. 2,3,6-trimethyl 21-β-cyclodextrin, sulphobutylether-β-cyclodextrin and hydroxypropyl-β-cyclodextrin in which the hydroxyl group on the hydroxypropyl substituent can be bonded to any one of the 3 carbon atoms making up the propyl group. Sulphobutylether-β-cyclodextrin is a relatively new compound and is available from Cydex, Overland Park, Kans.

A particularly preferred pharmaceutical excipient is 2-hydroxypropyl-β-cyclodextrin (HP-β-CD).

The concentration of the water solubility enhancing pharmaceutical excipient, e.g. cyclodextrin, in the liquid composition of the invention can be from 0.5 to 50% w/v, preferably from 0.5 to 20% w/v, more preferably from 1 to 20% w/v and particularly from 1 to 10% W/V.

By % w/v we mean the weight in grams of the pharmaceutical excipient, e.g. cyclodextrin, that is dissolved in 100 ml of water or other aqueous medium.

The concentration of the water solubility enhancing pharmaceutical excipient, e.g. cyclodextrin, in the solid formulation of the invention can be from 15 to 90% w/w, but is preferably from 30 to 75% w/w, more preferably from 45 to 60% w/w.

When the liquid composition of the present invention is intended for delivery into the nasal cavity or eye, it preferably comprises a gelling agent, or a bioadhesive material, or a material possessing both gelling and bioadhesive properties, to provide for controlled release of the fexofenadine in the nasal cavity. The release rate of the fexofenadine may be modified by changing the concentration of the gelling agent or bioadhesive material in the formulation.

By a bioadhesive material we mean a material that can interact with a mucosal surface such as that found in the nose or the eye. The bioadhesive effect may be achieved through the interaction of a positively charged polymer with the negatively charged surface of the cells lining the nasal mucosa or the corneal cells, or by the interaction of a positively charged polymer with the negative sugar group in mucin.

Suitable gelling agents for use in the compositions of the present invention include the polysaccharides, such as pectin, the alginates and gellan. These gelling agents are typically comprised in the liquid, particularly aqueous formulations of the invention at a concentration of from 0.1 to 20% w/v, i.e. from 0.1 to 20 g of the gelling agent per 100 ml of the liquid vehicle. Preferred compositions comprise from 0.5 to 10% w/v, e.g. from 1 to 10% w/v of the gelling agent.

Suitable gelling agents for use in liquid, particularly aqueous formulations also include gelling block copolymers. Suitable gelling block copolymers include the poloxamers such as Poloxamer 188, Poloxamer 237, Poloxamer 338, Poloxamer 407 and Poloxamer 427. These gelling materials are typically comprised in the liquid formulation at a concentration of from 1% to 30% w/v, preferably from 5 to 20%.

Suitable bioadhesive materials for the liquid composition of the invention include chitosan and the chitosan derivatives such as the trimethyl derivative.

A particularly suitable gelling agent in the liquid and particularly the aqueous formulations of the present invention is pectin which is able to significantly reduce the release/diffusion rate of fexofenadine hydrochloride from the formulation.

Pectins are materials which are found in the primary cell wall of all green land plants. They are heterogeneous materials, with a polysaccharide backbone that is uniform as α-1,4-linked polygalacturonic acid. Various neutral sugars have been identified in pectins such as xylose, galactose, rhamnose and arabinose.

Pectin can form gels in the presence of divalent ions such as calcium. The interaction of pectin with simulated nasal electrolyte solution can form a very strong gel, which can prolong the contact time of the formulation in the nasal cavity either through bioadhesive interactions and/or an increase in viscosity.

An important property of pectins is the extent to which the galacturonic acid groups are esterified. The degree of esterification (DE) of pectins found naturally can vary considerably (from 60 to 90%). The term DE is well understood by those skilled in the art and represents the percentage of the total number of galacturonic carboxyl groups which are esterified.

Pectins having a low DE, i.e. materials in which less than 50% and preferably less than 35% of the carboxyl groups are esterified, are particularly preferred. These can be prepared by the de-esterification of extracted pectins by way of an enzymatic process or by treatment with acid or ammonia in an alcoholic heterogeneous medium. Methods for the de-esterification of high DE pectins (which may be obtained from, for example, Sigma Fine Chemicals) are described in the article by Rollin in “Industrial Gums”, Academic Press, New York (1993) p. 257.

Pectins with a low DE can be obtained commercially from Copenhagen Pectin A/S as the commercial materials known as Slendid Type 100 and Slendid Type 110. These pectins have been extracted from citrus peel and standardised by the addition of sucrose. The degree of esterification is less than 50% for both pectins and is of the order of 10% for type 100 and 35% for type 110. Further materials include GENU pectin types LM1912CS and Pomosin pectin types LM12CG and LM18CG.

The concentration of pectin in the liquid formulation of the invention is preferably from 0.5 to 5% w/v.

A typical liquid composition for nasal delivery will comprise from 1 to 20 mg/ml of fexofenadine hydrochloride, from 1 to 200 mg/ml of hydroxypropyl-β-cyclodextrin and from 5 to 50 mg/ml of pectin. A preferred liquid composition will comprise 10 mg/ml of fexofenadine hydrochloride, 100 mg/ml of hydroxypropyl-β-cyclodextrin and 10 mg/ml of pectin.

The compositions of the invention can be prepared in accordance with known techniques.

For example, an aqueous composition can be prepared by dissolving or dispersing the fexofenadine and pharmaceutical excipient in water. Compositions containing pectin can be prepared by dissolving or dispersing the fexofenadine, pharmaceutical excipient and pectin in water, optionally together with simple monovalent electrolytes such as NaCl to provide isotonicity, agents such as glycerol and preservatives such as sodium metabisulphate.

The composition of the invention can also be a powder formulation. Compositions of this type can be prepared by solubilising the fexofenadine in an aqueous solution of a solid excipient which increases the solubility of the fexofenadine in water, preferably cyclodextrin, and recovering the fexofenadine/excipient mixture by removing the water, e.g. by oven drying or freeze drying.

Optionally, a gelling/bioadhesive material can be included in the powder formulation. This material can be added to the drug/excipient mixture either prior to or after drying. Suitable gelling/bioadhesive materials which may be used, e.g. in microsphere form, include starch, chitosan, polyvinyl pyrrolidone, alginate, polycarbophil, pectin, hyaluronic acid (and esters thereof), agar, agarose, dextran, ovalbumin, collagen and casein, with starch and chitosan being preferred, especially starch. Where a gelling/bioadhesive material is employed, the concentration of this material will typically be in the range of from 5 to 80% w/w, preferably in the range of from 15 to 65% w/w and more preferably in the range of from 20 to 50% w/w.

As a compromise between solubility and acceptability for administration to mucosal surfaces, a pH of 3 to 9 is preferred for the composition, with a pH of 4 to 8 being especially preferred.

The present formulation may be administered to the nose of a patient using a spray device, such as those supplied by Valois and Pfieffer. These devices may be single dose or multiple dose systems. The present formulation may also be administered to the eye of a patient using an eye dropper. For such an ophthalmic product a thickening agent may be added such as polyvinylalcohol or hypromellose.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown.

In the drawings:

FIG. 1 is a schematic cross-sectional view of a Franz diffusion cell.

FIG. 2 is a schematic representation of a Franz diffusion cell arranged in a closed loop circuit.

FIG. 3 shows the cumulative release/diffusion of fexofenadine hydrochloride from two formulations, HP-β-CD and pectin/HP-β-CD, into simulated nasal electrolyte solution.

### DETAILED DESCRIPTION OF THE INVENTION

The Franz diffusion cell depicted in FIG. 1 is known in the art. The cell (1) comprises a sample compartment (2), a membrane (3) that supports the formulation being tested, a flange cap (4) which locates on the membrane, a metal clasp (5) which secures the flange cap and membrane in place, a water jacket (6), an eluant inlet (7) which leads from a peristaltic pump, an eluant outlet (8) which leads to a flow-through cuvette and a receptor compartment (9) with a stirrer (10) where eluant is circulated via the peristaltic pump to the cuvette which locates in a UV spectrophotometer.

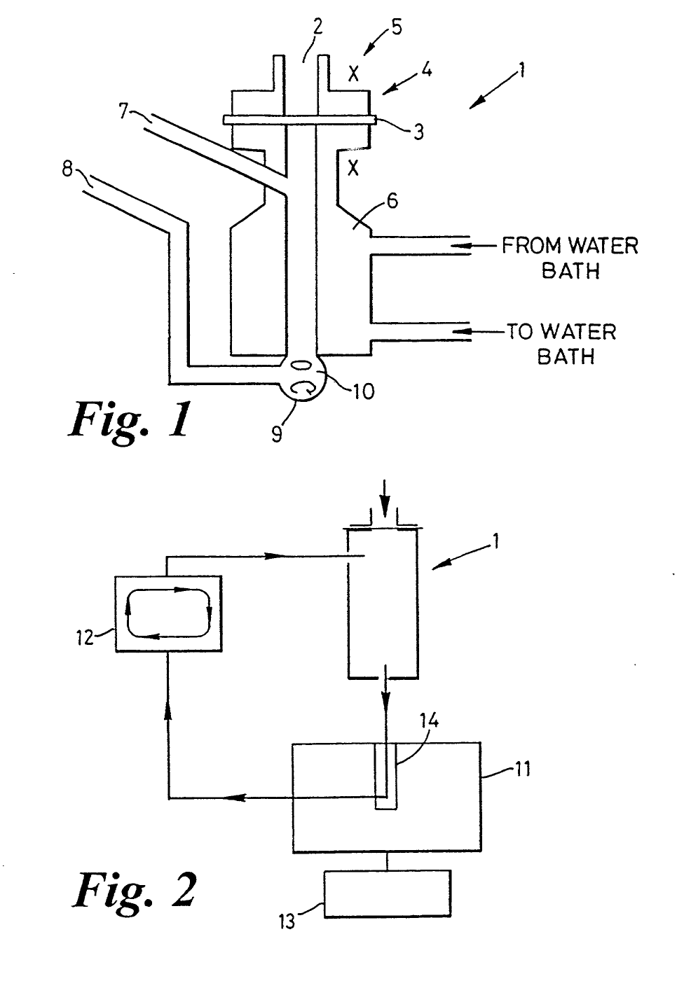

In the closed loop circuit depicted in FIG. 2, the Franz diffusion cell (1) is connected in a circuit comprising a UV spectrophotometer (11), a peristaltic pump (12) and a printer (13). The flow through cuvette (14) locates in the UV spectrophotometer (11). The sample being analysed is charged to the apparatus as shown by the emboldened arrow.

The present invention is now illustrated but not limited with reference to the following examples.

### EXAMPLE 1

### Analytical Methods for Fexofenadine

A UV method for quantifying fexofenadine hydrochloride in water at pH 4.0 was established for measuring the solubility of fexofenadine hydrochloride in water.

A solution of 1 mg/ml fexofenadine hydrochloride (Hoechst Marion Roussel) in water was prepared and the pH of the solution was adjusted to 4.0 with 0.5 M sodium hydroxide solution. Phthalate buffer pH 4.0 was also prepared. Both solutions were scanned using a Hewlett Packard 8452A Diode Array Spectrophotometer. An absorbance wavelength of 260 nm was selected to prepare a calibration curve for fexofenadine hydrochloride in water. Phthalate buffer pH 4.0 had strong UV absorbance between 190 and 320 nm and was not a suitable medium for the drug.

A series of solutions of fexofenadine hydrochloride prepared in water at concentrations of 150, 300, 450, 600 and 750 μg/ml and adjusted to pH 4.0 with hydrochloric acid or sodium hydroxide were assayed at 260 nm using the Hewlett Pachard 8452A Diode Array Spectrophotometer. The calibration equation was as follows: Y=816.284 X−3.960 (r=1.000, where Y is the drug concentration in mg/ml and X is the UV absorbance (linearity over 150 to 750 μg/ml)).

### EXAMPLE 2

### UV Method Validation for Analysis of Fexofenadine Hydrochloride in Cyclodextrin Solutions at pH 4.0

Two cyclodextrins, α-cyclodextrin (α-CD) and hydroxy propyl-β-cyclodextrin (HP-β-CD), were assessed for their effect on fexofenadine hydrochloride solubility. It was intended that the UV method would be used to measure the solubility of fexofenadine hydrochloride in cyclodextrin solutions at pH 4.0. First the UV absorbance of α-CD and HP-β-CD was investigated to establish whether they interfere with analysis of the drug.

Solutions of 100 mg/ml α-CD and 100 mg/ml HP-β-CD at pH 4.0 were prepared and UV scanned. Solutions at pH 4.0 and containing fexofenadine hydrochloride at concentrations of 150, 450 and 750 μg/ml in water were prepared and assayed by the UV method at 260 nm.

At 260 nm, the UV absorbance of 150, 450 and 750 μg/ml fexofenadine hydrochloride in water was 0.1900, 0.5612 and 0.9122 respectively, but the absorbance of 100 mg/ml α-CD and 100 mg/ml HP-β-CD was 0.0239 and 0.0832 respectively. The absorbance of fexofenadine hydrochloride solution was affected little by the presence of α-CD and the UV method is valid to assay the concentration of the drug in α-CD solutions. The 100 mg/ml HP-β-CD caused a minor interference at 260 nm. However, in an actual formulation, the UV absorbance of HP-β-CD would be minimal compared to that of fexofenadine hydrochloride and therefore the UV method can also be used to assay the concentration of the drug in HP-β-CD solutions.

### EXAMPLE 3

### Solubility of Fexofenadine Hydrochloride in Water and Cyclodextrin Solutions at pH 4.0

a) The Solubility of Fexofenadine Hydrochloride in Water at pH 4.0

An aqueous suspension containing 10 mg/ml fexofenadine hydrochloride at pH 4.0 was stirred for 24 hours at room temperature. The mixture was centrifuged and the supernatant was passed through a 0.45 μm membrane filter to remove drug particles. The filtered solution was assayed by the UV method at 260 nm.

b) The Solubility of Fexofenadine Hydrochloride in Cyclodextrin Solutions at pH 4.0

α-CD and HP-β-CD aqueous solutions were prepared at concentrations of 10, 25, 50 and 100 mg/ml respectively. To 10 ml of each solution, 100 mg of fexofenadine hydrochloride was added, stirred and the pH of the solutions was adjusted to pH 4.0 by adding hydrochloric acid or sodium hydroxide. If the drug dissolved completely, a further 100 mg of fexofenadine hydrochloride was added. The suspensions were stirred for 24 hours and centrifuged. The supernatants were filtered through a 0.45 m membrane filter to remove drug particles, then diluted and assayed by the UV method at 260 nm.

The solubility of fexofenadine hydrochlorides in water, α-CD and HP-β-CD solutions is listed in Table 1. The solubility of fexofenadine hydrochloride in water is 0.6 mg/ml. The solubility in aqueous solution was increased by both α-CD and HP-β-CD, and the enhancement of the solubility depended on the concentration of cyclodextrin in aqueous solution. The higher the concentration of cyclodextrin in solution, the higher the solubility of the drug that was obtained. HP-β-CD improved the solubility much more than α-CD. While not wishing to be bound by any theory, we believe that this increased solubility for fexofenadine in HP-β-CD is due to the fact that fexofenadine can complex more efficiently with this cyclodextrin and perhaps fit better inside the cyclodextrin molecule. A linear relationship of fexofenadine hydrochloride solubility increasing with the concentrations of α-CD and HP-β-CD was found. It can be predicted that a higher solubility of fexofenadine hydrochloride in aqueous solution will be achieved with a higher concentration of HP-β-CD.

TABLE 1
The solubility of fexofenadine hydrochloride in aqueous solutions at pH 4.

| Solution | Solubility of fexofenadine hydrochloride (mg/ml) |
|---|---|
| Water | 0.6 |
| α-CD | |
| 10 mg/ml | 0.6 |
| 25 mg/ml | 1.2 |
| 50 mg/ml | 2.7 |
| 100 mg/ml | 3.3 |
| HP-β-CD | |
| 10 mg/ml | 1.9 |
| 25 mg/ml | 3.5 |
| 50 mg/ml | 8.1 |
| 100 mg/ml | 13.1 |

The molecular weights of fexofenadine hydrochloride, α-CD and HP-β-CD are 538, 972 and 1135 respectively. At a solubility of 3.3 mg/ml fexofenadine hydrochloride in 100 mg/ml α-CD aqueous solution, the weight ratio of fexofenadine hydrochloride: α-CD is 1:30.3, which is equal to a molar ratio of 1:16.8. At a solubility of 13.1 mg/ml fexofenadine hydrochloride in 100 mg/ml HP-β-CD aqueous solution, the weight ratio of fexofenadine hydrochloride: HP-β-CD is 1:7.6, which is equal to a molar ratio of 1:3.6.

### EXAMPLE 4

### A Pectin Gelling Formulation for Controlled Release of Fexofenadine Hydrochloride

The feasibility of producing a gelling formulation for controlled release of fexofenadine hydrochloride was investigated.

Formulation 1: 10 mg/ml fexofenadine+100 mg/ml HP-β-CD

2 g of HP-β-CD was dissolved in 18-19 ml of water in a 20 ml volumetric flask. 200 mg of fexofenadine hydrochloride was added to the solution and stirred until the drug had dissolved. The pH of the solution was adjusted to 4.0 by the addition of hydrochloric acid or sodium hydroxide, then the solution was made up to volume with water.

Formulation 2: 10 mg/ml fexofenadine+100 mg/ml HP-β-CD+10 mg/ml pectin

50 mg of pectin was dissolved in 5 ml of Formulation 1 in a 5 ml volumetric flask.

Preparation of simulated nasal electrolyte solution:

8.77 g of sodium chloride, 2.98 g of potassium chloride and 0.59 g of calcium chloride dihydrate were dissolved in 1 liter of water in a 1 liter volumetric flask.

Release/diffusion testing:

A Franz diffusion cell apparatus was set up in a closed loop circuit. FIG. 1 shows the cell and FIG. 2 shows the cell arranged in a closed loop circuit. The operating parameters are listed below.

Medium: Simulated nasal electrolyte solution

Medium temperature: 37° C.

Membrane: Cellulose nitrate, 0.45 μm pore size

Volume of the closed loop arrangement: 8.8 ml

Stirring speed of a magnetic stirrer: 4

Peristaltic pump flow rate: 1 (The Cole-Parmer Masterflex peristaltic pump, Model 7518-60, fitted with Masterflex 14 silicone tubing)

Sample volume: 0.4 ml (contained 4 mg of fexofenadine hydrochloride, the maximum concentration of the drug in medium will be around 450 μg/ml)

Drug analysis: UV at 260 nm

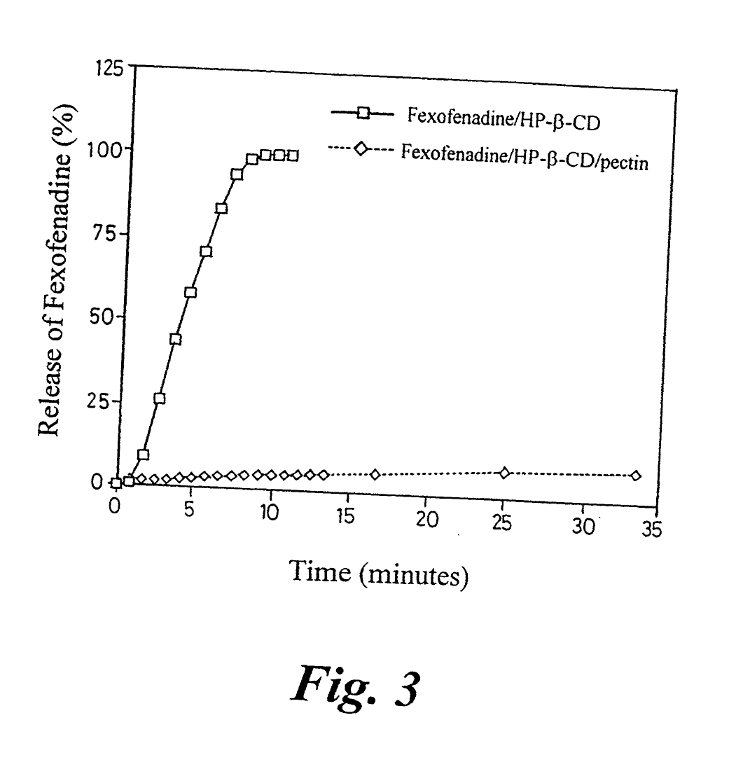

Formulation 2 interacted with simulated nasal electrolyte solution and formed a strong gel when it was applied on the membrane of the diffusion apparatus. FIG. 3 shows the cumulative release/diffusion of fexofenadine hydrochloride from two formulations, HP-β-CD and pectin/HP-β-CD, into simulated nasal electrolyte solution. The maximum UV absorbance of Formulation 1 (control) reached during the diffusion experiment represented 100% drug release and was used to calculate the percentage of release at each selected time point. The release/diffusion rate of fexofenadine hydrochloride from pectin/HP-β-CD solution was significantly slower than from the HP-β-CD solution. As a control solution, fexofenadine hydrochloride diffused through the membrane very rapidly with complete drug release in 10 minutes. However, after 30 minutes, less than 10% of the drug had been released from the pectin containing formulation.

These examples show the solubility of fexofenadine hydrochloride in aqueous solution at pH 4.0 was improved significantly using cyclodextrins. The enhancement of fexofenadine hydrochloride solubility in aqueous solution depends on the concentration of cyclodextrin. HP-β-CD increased the solubility much more than α-CD. The solubilities in water, 100 mg/ml α-CD and 100 mg/ml HP-β-CD aqueous solutions at pH 4.0 were 0.6, 3.3, and 13.1 mg/ml, respectively. A pectin gelling formulation containing 10 mg/ml fexofenadine hydrochloride and 100 mg/ml HP-β-CD showed very slow release of the drug which forms the basis of a controlled release formulation for nasal administration of fexofenadine.

The formulation described in Example 4 can be administered to the nose of a patient using a spray device. Such devices can be obtained from companies such as Valois and Pfieffer and may be single dose or multiple dose systems.

Similarly an ophthalmic formulation can be prepared in the same manner as in Example 4 and administered to the eye using an eye dropper. For such an ophthalmic product a thickening agent can be added such as polyvinylalcohol or hypromellose.

### EXAMPLE 5

### Cosolvent (Water/Propylene Glycol) Formulation Containing 10 mg/ml Fexofenadine Hydrochloride

250 mg of fexofenadine hydrochloride was weighed into a 5 ml volumetric flask. To the flask was added 4 ml of propylene glycol (1,2-propanediol) (Sigma, Poole, UK) and the contents stirred until the drug had dissolved. The flask contents were made up to 5 ml with propylene glycol (final drug concentration=50 mg/ml). Into a 10 ml volumetric flask was transferred 2 ml of the 50 mg/ml fexofenadine hydrochloride solution. The flask contents were made up to 10 ml with water to form a solution containing 10 mg/ml fexofenadine hydrochloride.

### EXAMPLE 6

### Cosolvent (Water/Tetraglycol) Formulation Containing 10 mg/ml Fexofenadine Hydrochloride and 5 mg/ml Chitosan Glutamate

250 mg of fexofenadine hydrochloride was weighed into a 5 ml volumetric flask. To the flask was added 4 ml of tetraglycol (glycofurol) (Sigma) and the contents stirred until the drug had dissolved. The flask contents were made up to 5 ml with tetraglycol (final drug concentration=50 mg/ml). Into a 10 ml volumetric flask were added 100 mg of chitosan glutamate and 8 ml of water. The flask contents were stirred until the chitosan had dissolved and then made up to 10 ml with water (final concentration=10 mg/ml chitosan glutamate). In a 10 ml volumetric flask were mixed 2 ml of the 50 mg/ml fexofenadine hydrochloride solution and 5 ml of the 10 mg/ml chitosan glutamate solution. The flask contents were made up to 10 ml with water to form a solution containing 10 mg/ml fexofenadine hydrochloride and 5 mg/ml chitosan glutamate.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A composition comprising (i) fexofenadine or a pharmaceutically acceptable salt thereof and (ii) a pharmaceutical excipient which increases the solubility of the fexofenadine or salt in water, which is adapted for delivery of the fexofenadine or pharmaceutically acceptable salt thereof to the eye or nose.

2. A composition as claimed in claim 1 for use in medicine.

3. A composition as claimed in claim 1, wherein the pharmaceutical excipient is a water miscible, non-aqueous solvent.

4. A composition as claimed in claim 3, wherein the solvent is propylene glycol or glycofurol (tetraglycol).

5. A composition as claimed in claims 1, wherein the pharmaceutical excipient is a material which is able to complex with the fexofenadine or pharmaceutically acceptable salt thereof.

6. A composition as claimed in claims 1, wherein the pharmaceutical excipient is a cyclodextrin.

7. A composition as claimed in claim 6, wherein the cyclodextrin is hydroxypropyl-β-cyclodextrin.

8. A composition as claimed in claim 1, which further comprises a material that provides for controlled release of the fexofenadine or pharmaceutically acceptable salt thereof.

9. A composition as claimed in claims 1, which further comprises a gelling agent or bioadhesive material.

10. A composition as claimed in claim 9, wherein the gelling agent or bioadhesive material is a polysaccharide.

11. A composition as claimed in claim 10, wherein the gelling agent or bioadhesive material is selected from the group consisting of pectin, alginate, starch, gellan and chitosan.

12. A composition as claimed in claim 9, wherein the gelling agent is a block co-polymer.

13. A composition as claimed in claim 12, wherein the block co-polymer is a poloxamer.

14. A composition as claimed in claim 1, which is an aqueous composition additionally comprising an aqueous vehicle.

15. The use of a composition according to claim 1, in the manufacture of a medicament for administration of fexofenadine or a pharmaceutically acceptable salt thereof to the nose or to the eye.

16. The use of a composition according to claim 1, in the manufacture of a medicament for the treatment of rhinitis.

17. The use of (i) fexofenadine or a pharmaceutically acceptable salt thereof and (ii) a pharmaceutical excipient which increases the solubility of the fexofenadine or salt in water in the manufacture of a medicament for administration of the fexofenadine or salt to the nose or to the eye.

18. The use of a composition according to claim 9, in the manufacture of a medicament for controlling the release of fexofenadine or a pharmaceutically acceptable salt thereof when the composition is administered to the nose or to the eye.

19. The use of a composition according to claim 9, in the manufacture of a medicament for treating rhinitis by the controlled release of the fexofenadine or a pharmaceutically acceptable salt thereof.

20. A method of treating a patient in need of treatment with fexofenadine or a pharmaceutically acceptable salt thereof which comprises administering an effective amount of a composition according to claim 1 to a patient in need of such treatment.

21. A method of treating rhinitis which comprises administering an effective amount of a composition according to claim 1, to a patient in need of such treatment.

22. A method of treating a patient with a controlled release dose of fexofenadine or a pharmaceutically acceptable salt thereof which comprises administering an effective amount of a composition according to claim 9, to a patient in need of such treatment.
